(19) United States
(12) Patent Application Publication
(10) Pub. No.: US 2019/0343780 A1
(43) Pub. Date: Nov. 14, 2019

(54) LOW DOSE DRUG COMBINATIONS FOR USE IN PREVENTING AND TREATING NEURONAL DAMAGE

(71) Applicant: RVX THERAPEUTICS LTD., Kiriat Yam (IL)

(72) Inventors: Sergey SERDYUK, Jerusalem (IL); Vladmir RITTER, Kiriat Yam (IL)

(21) Appl. No.: 16/472,017

(22) PCT Filed: Dec. 14, 2017
(86) PCT No.: PCT/IL2017/051348

§ 371 Date: Jun. 20, 2019

Related U.S. Application Data
(60) Provisional application No. 62/437,692, filed on Dec. 22, 2016.

(51) Int. Cl. A61K 31/133; A61K 31/13; A61P 25/28

(52) U.S. Cl. CPC A61K 31/133; A61P 25/28; A61K 31/13

(57) ABSTRACT

The present invention provides low-dose, synergistic combinations of NMDA receptor antagonists and peripheral adrenergic receptor agonists, and methods for their use in preventing and treating hypoxia and neuronal damage.

### FIELD OF THE INVENTION

The present invention relates to pharmaceutical compositions comprising beneficial combinations of NMDA receptor antagonists and peripheral adrenergic receptor agonists, and to methods for their use in preventing and treating hypoxia and neuronal damage.

### BACKGROUND OF THE INVENTION

Stroke is a situation in which lower-than-normal blood flow to the brain results in neuronal cell death. There are two main types of stroke: ischemic stroke, which is caused by a lack of blood flow, and hemorrhagic stroke, which is caused by bleeding. In both cases, the brain does not function properly. Signs and symptoms of a stroke, which often appear soon after the stroke has already occurred, can include an inability to move or feel on one side of the body or loss of vision to one side, among others. If symptoms last less than one or two hours after the stroke, the episode is known as a transient ischemic attack (TIA). The main risk factor for stroke is high blood pressure. Other risk factors include tobacco smoking, obesity, high blood cholesterol, diabetes mellitus, previous TIA, and atrial fibrillation. An ischemic stroke is typically caused by blockage of a blood vessel.

In the first hours of an ischemic stroke, blood pressure drops significantly, developing post-stroke hypotension and cerebral hypo-perfusion which progressively increases in the next few days. As a result of the fall in blood pressure, the total cerebral blood flow is decreasing, collateral blood flow around the infarct area reduces, and ischemia increases, leading to impaired recovery of neurological and cognitive functions. It is also known that 30-50% of stroke patients develop orthostatic hypotension and orthostatic cerebral hypo-perfusion, which aggravate the course of stroke and hinder rehabilitation after a stroke.

Induced hypertension by intravenous injection of phenylephrine during the first 12 hours after stroke leads to a rapid increase in blood pressure (from 100-110 mm to 160-170 mm) and a significant improvement in cerebral blood flow, especially in the collateral circulation around the infarct zone, and also causes a significant recovery of neurological and cognitive functions 1 to 6 days after stroke. (Hillis et al., Cerebrovasc. Dis., 2003, Vol. 16(3), pages 236-246). The disadvantage of induced hypertension is that it does not increase the therapeutic window for thrombolytic agents (used to dissolve blood clots) and can effectively eliminate already evolved neurodegenerative changes in neurons, because it does not cause significant neuroprotective effect (Bogoslovsky et al., BMC Neurol., 2006, Vol. 6(46)). However, Induced hypertension is further not recommended for stroke prophylaxis and prevention of transient ischemic attacks, preceding stroke.

It should be noted that the use of antihypertensive drugs for the prevention of stroke is only justified in patients with high blood pressure without deregulated vascular tonus. However, 50-70% of the elderly patients have disrupted sympathetic regulation of vascular tone and develop orthostatic hypotension and orthostatic syncope, leading to the development of cerebral hypoperfusion and cerebral vasospasm, contributing to the emergence of transient ischemic attack and stroke (Eigenbrodt et al., Stroke, 2000, Vol. 31(10), pages 2307-2313). More, high comorbidity of orthostatic hypotension and ischemic stroke, and transient ischemic attacks, reaching 30% in the elderly patients has been reported (Chou et al., Int. J. Cardiol., 2015, Vol. 15(195), pages 40-44). In 10-15% of the cases, ischemic stroke is directly preceded by orthostatic syncope (Ryan et al., Age Ageing, 2015, Vol. 44(4), pages 655-661). The use of antihypertensive drugs in these patients increases the orthostatic hypotension and increase the risk of stroke.

In a focal cerebral ischemia model in rats, intravenous administration of phenylephrine before the ligation of the middle cerebral artery prevented infarction development and reduced neurological damage (Drummond et al., Stroke, 1989, Vol. 20(11), pages 1538-1544), which indicates the possibility of phenylephrine utilization for the prevention of stroke. It should be noted, however, that midodrine and phenylephrine are used for the prevention of cerebral ischemia in high doses and significantly increase blood pressure, which is highly undesirable in patients with hypertension and may even contribute to the development of stroke. Therefore, there remains a need for decreased effective doses of pressor agonists with a profound anti-ischemic effect, without significantly increasing blood pressure.

In a paper by Culmsee and coworkers (Culmsee et al., Stroke, 2004, Vol. 35(5), pages 1197-1202), prophylactic administration of memantine at a high dose of 20 mg/kg in a focal cerebral ischemia model in rats prevented injury of the brain and reduced neurological disorders, indicating potential use of memantine in stroke prevention. In a paper by Kafi and coworkers (Kafi et al., Iran J. Pharm. Res., 2014, Vol. 13(2), pages 591-598), memantine was administered orally starting within 12 hours after ischemic stroke in the high dose of 20 mg, and continued to be administered in same dose three times a day for 5 days as neuroprotective therapy. When administered at a high dose of 20 mg for 6 months memantine significantly improved neurological and cognitive function patients in the subacute phase and recovery phase of rehabilitation of the stroke (Litvinenko et al., Zh Nevrol Psikhiatr Im S S Korsakova. 2013, Vol. 113(9):29-35). However, prolonged use at a high dose of memantine is accompanied with significant side effects such as development of agitation, insomnia, irritability, and psychosis (Gmiro et al., Eksp Klin Farmakol., 2000, Vol. 63(6), pages 3-8). Thus, it remains desirable to reduce the effective dose of memantine, which may significantly reduce its side effects.

International Patent Application Publication No. WO 2008/018084 relates to therapeutic uses of active agents derived from human plasminogen activator inhibitor 1 (PAI-1) for preventing neuronal damage due particularly to hypoxic or thromboembolic stroke and brain injury.

Further limitations and disadvantages of conventional and traditional approaches will become apparent to one of skill in the art, through comparison of such systems with some aspects of the present invention. There remains a need for new, efficient, low-dose therapies for preventing and treating hypoxia and neuronal damage.

### SUMMARY OF THE INVENTION

The present invention relates to novel compositions and methods for preventing and treating neuronal damage of different etiologies and mechanisms, based on low-dose, synergic combinations of N-methyl-D-aspartate (NMDA) receptor antagonists and peripheral adrenergic receptor agonists. Specifically, the present invention provides compositions and methods for preventing and treating stroke-related neuronal damages by systemic administration of such combinations. The present invention is based on the surprising findings that NMDA receptor antagonists, such as memantine, and peripheral adrenergic receptor agonists, such as phenylephrine, known to have a neuroprotective effect only in high dosages, have a comparable effect when combined in much lower dosages. The novel use of such low-dose combinations was further surprisingly found to be substantially free from the adverse side-effects which often accompany the use of standard, high-dosage, stand-alone, neuroprotective drugs.

The present invention provides, in one aspect, a method for preventing, ameliorating the progression or treating neuronal damage in a subject in need thereof, the method comprising systemically administering to the subject: (i) at least one N-methyl-D-aspartate (NMDA) receptor antagonist, and (ii) at least one peripheral adrenergic receptor agonist, wherein the at least one NMDA receptor antagonist and the at least one peripheral adrenergic receptor agonist are administered in a molar ratio of about 25:1 to about 500:1, respectively.

In certain embodiments, the neuronal damage is associated with cerebral hypoxia, cerebral ischemia, or over-stimulation of an ionotropic neuronal glutamate receptor. In certain embodiments, the cerebral hypoxia is selected from the group consisting of hemic hypoxia, asphyctic hypoxia, and any combination thereof. In certain embodiments, the ionotropic neuronal glutamate receptor is selected from the group consisting of an NMDA receptor, an α-amino-3-hydroxy-5-methyl-4-isoxazolepropionic acid (AMPA) receptor, a kainate receptor (KAR), and any combination thereof. Each possibility represents a separate embodiment of the invention.

In certain embodiments, the neuronal damage is associated with at least 30% weight loss compared to his weight prior to the neuronal damage, a neurological disturbance, or premature death. In certain embodiments, the subject has been diagnosed with stroke, chronic cerebral ischemia, Alzheimer's disease (AD), multiple sclerosis (MS), progressive supranuclear palsy (PSP), Parkinson disease (PD), Huntington's chorea, amyotrophic lateral sclerosis, spinal trauma, brain trauma, spinal inflammation or brain inflammation. In certain embodiments, the subject is in increased risk to suffer a neuronal damage. In certain embodiments, the subject has experienced or is experiencing a condition selected from the group consisting of stroke, chronic cerebral ischemia, chronic cerebral hypoxia, hypoxic hypotension, cerebral hypo-perfusion and syncope. In certain embodiments, the subject has suffered a neuronal damage. In certain embodiments, the subject has experienced a condition selected from the group consisting of stroke, chronic cerebral ischemia, chronic cerebral hypoxia, hypoxic hypotension, cerebral hypo-perfusion and syncope. Each possibility represents a separate embodiment of the invention.

In certain embodiments, the at least one NMDA receptor antagonist and the at least one peripheral adrenergic receptor agonist are administered in a molar ratio of about 25:1 to about 95:1, about 30:1 to about 50:1, about 45:1 to about 95:1, about 90:1 to about 180:1, about 125:1 to about 180:1, about 125:1 to about 500:1, about 130:1 to about 180:1, or about 250:1 to about 500:1. Each possibility represents a separate embodiment of the invention.

In certain embodiments, the at least one NMDA receptor antagonist is selected from the group consisting of an uncompetitive channel blocker, a competitive antagonist, a non-competitive antagonist, and a glycine antagonist. In certain embodiments, the at least one NMDA receptor antagonist is an uncompetitive channel blocker. In certain embodiments, the uncompetitive channel blocker is memantine.

In certain embodiments, the at least one peripheral adrenergic receptor agonist is selected from the group consisting of a non-selective agonist of a plurality of adrenergic receptors and a selective agonist of α1 adrenergic receptor.

In certain embodiments, the at least one peripheral adrenergic receptor agonist is a non-selective agonist of a plurality of adrenergic receptors. In certain embodiments, the at least one peripheral adrenergic receptor agonist is epinephrine.

In certain embodiments, the at least one NMDA receptor antagonist and the at least one peripheral adrenergic receptor agonist are administered in a molar ratio of about 90:1 to about 500:1. In certain embodiments, the at least one NMDA receptor antagonist and the at least one peripheral adrenergic receptor agonist are administered in a molar ratio of about 90:1 to about 180:1, about 125:1 to about 180:1, about 125:1 to about 500:1, about 130:1 to about 180:1, or about 250:1 to about 500:1. In certain embodiments, the at least one NMDA receptor antagonist and the at least one peripheral adrenergic receptor agonist are administered in a molar ratio of about 125:1 to about 180:1, about 130:1 to about 180:1, about 125:1 to about 500:1, or about 250:1 to about 500:1. In certain embodiments, the at least one NMDA receptor antagonist is memantine. Each possibility represents a separate embodiment of the invention.

In certain embodiments, the at least one peripheral adrenergic receptor agonist is a selective agonist of α1 adrenergic receptor. In certain embodiments, the at least one peripheral adrenergic receptor agonist is phenylephrine.

In certain embodiments, the at least one NMDA receptor antagonist and the at least one peripheral adrenergic receptor agonist are administered in a molar ratio of about 25:1 to about 95:1. In certain embodiments, the at least one NMDA receptor antagonist and the at least one peripheral adrenergic receptor agonist are administered in a molar ratio of about 25:1 to about 95:1, about 30:1 to about 50:1, or about 45:1 to about 95:1. In certain embodiments, the at least one NMDA receptor antagonist and the at least one peripheral adrenergic receptor agonist are administered in a molar ratio of about about 30:1 to about 50:1 or about 45:1 to about 95. In certain embodiments, the at least one NMDA receptor antagonist is memantine. Each possibility represents a separate embodiment of the invention.

In certain embodiments, the systemic administration is selected from the group consisting of oral, intraperitoneal and intramuscular administration. Each possibility represents a separate embodiment of the invention.

In certain embodiments, the at least one NMDA receptor antagonist and the at least one peripheral adrenergic receptor agonist are comprised in the same pharmaceutical composition. In certain embodiments, the at least one NMDA receptor antagonist and the at least one peripheral adrenergic receptor agonist are comprised in different pharmaceutical compositions. In certain embodiments, the at least one NMDA receptor antagonist and the at least one peripheral adrenergic receptor agonist are administered at separate times. In certain embodiments, the at least one NMDA receptor antagonist and the at least one peripheral adrenergic receptor agonist are administered concomitantly.

The present invention further provides, in another aspect, a method for treating at least one symptom of transient ischemic attack or ischemic stroke in a subject in need thereof, comprising administering to the subject a pharmaceutical composition by systemic administration, the pharmaceutical composition comprising: (i) at least one N-methyl-D-aspartate (NMDA) receptor antagonist, and (ii) at least one peripheral adrenergic receptor agonist, wherein the at least one NMDA receptor antagonist and the at least one peripheral adrenergic receptor agonist are administered in a molar ratio of about 25:1 to about 500:1, respectively.

The present invention further provides, in another aspect, a method for treating at least one symptom of cerebral hypoxia in a subject in need thereof, comprising administering to the subject a pharmaceutical composition by systemic administration, the pharmaceutical composition comprising: (i) at least one N-methyl-D-aspartate (NMDA) receptor antagonist, and (ii) at least one peripheral adrenergic receptor agonist, wherein the at least one NMDA receptor antagonist and the at least one peripheral adrenergic receptor agonist are administered in a molar ratio of about 25:1 to about 500:1, respectively.

In certain embodiments of the methods described above, the at least one symptom is neuronal damage.

The present invention further provides, in another aspect, a pharmaceutical composition comprising: (i) at least one NMDA receptor antagonist, and (ii) at least one peripheral adrenergic receptor agonist, wherein the at least one NMDA receptor antagonist and the at least one peripheral adrenergic receptor agonist are in a molar ratio of about 25:1 to about 500:1, respectively.

In certain embodiments, the pharmaceutical composition described above is for use in a method for preventing, ameliorating the progression or treating neuronal damage. In certain embodiments, the pharmaceutical composition described above is for use in a method for preventing, ameliorating the progression or treating at least one symptom of transient ischemic attack or ischemic stroke. In certain embodiments, the pharmaceutical composition described above is for use in a method for preventing, ameliorating the progression or treating at least one symptom of cerebral hypoxia. In certain embodiments, the use comprises administering the pharmaceutical composition by systemic administration.

The present invention further provides, in another aspect, a use of a pharmaceutical composition as described above in the manufacture of a medicament for (i) preventing, ameliorating the progression or treating neuronal damage, (ii) treating at least one symptom of transient ischemic attack or ischemic stroke, or (iii) treating at least one symptom of cerebral hypoxia.

The present invention further provides, in another aspect, a use of a pharmaceutical composition as described above in the manufacture of a medicament for treating at least one symptom of Alzheimer's disease (AD), multiple sclerosis (MS), progressive supranuclear palsy (PSP), Parkinson disease (PD), Huntington's chorea, amyotrophic lateral sclerosis, spinal trauma, brain trauma, spinal inflammation or brain inflammation.

The present invention further provides, in another aspect, a kit comprising: (i) at least one NMDA receptor antagonist, and (ii) at least one peripheral adrenergic receptor agonist, wherein the at least one NMDA receptor antagonist and the at least one peripheral adrenergic receptor agonist are in a molar ratio of about 25:1 to about 500:1.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The present invention relates to compositions and methods for preventing and treating neuronal damage of different etiologies and mechanisms. Such methods are based on low-dose, synergic combinations of N-methyl-D-aspartate (NMDA) receptor antagonists and peripheral adrenergic receptor agonists. More specifically, the present invention provides methods for preventing and treating stroke-related neuronal damages by systemic administration of such combinations. The present invention is based on the surprising findings that NMDA receptor antagonists and peripheral adrenergic receptor agonists which are known to have a neuroprotective effect only in high dosages, which are now considered standard, have at least the same effect when combined in much lower dosages. It was also surprisingly found that the use of much-lower-than-standard drug dosages is substantially free from neuroprotective drugs-related side-effects.

The present invention provides, in an aspect, a method for preventing neuronal damage, preventing the progression of neuronal damage or treating neuronal damage in a subject in need thereof, comprising administering to the subject a pharmaceutical composition by systemic administration, the pharmaceutical composition comprising (i) at least one N-methyl-D-aspartate (NMDA) receptor antagonist, and (ii) at least one peripheral adrenergic receptor agonist, wherein the at least one NMDA receptor antagonist and the at least one peripheral adrenergic receptor agonist are administered in a molar ratio of at least about 25 to 1, respectively.

The present invention provides, in another aspect, a method for preventing, ameliorating the progression or treating neuronal damage in a subject in need thereof, the method comprising systemically administering to the subject: (i) at least one N-methyl-D-aspartate (NMDA) receptor antagonist, and (ii) at least one peripheral adrenergic receptor agonist, wherein the at least one NMDA receptor antagonist and the at least one peripheral adrenergic receptor agonist are administered in a molar ratio of about 25:1 to about 500:1, respectively.

In certain embodiments, the neuronal damage is cerebral neuronal damage. In certain embodiments, the neuronal damage is spinal neuronal damage. In certain embodiments, the neuronal damage is cerebral and spinal neuronal damage.

In certain embodiments, the neuronal damage is associated with cerebral hypoxia, hypoxic hypoxia, hypoxemic hypoxia, hemic hypoxia, histotoxic hypoxia, asphyctic hypoxia, hypemic hypoxia, ischemic hypoxia, ischemic stroke, hemorrhagic stroke, transient ischemic attack, acute forms thereof and/or transient forms thereof. Each possibility represents a separate embodiment of the invention.

In certain embodiments, the neuronal damage is associated with cerebral hypoxia. In certain embodiments, the neuronal damage is associated with cerebral ischemia. In certain embodiments, the neuronal damage is associated with over-stimulation of an ionotropic neuronal glutamate receptor. In certain embodiments, the cerebral hypoxia is hemic hypoxia. In certain embodiments, the cerebral hypoxia is asphyctic hypoxia. In certain embodiments, the ionotropic neuronal glutamate receptor is selected from the group consisting of an NMDA receptor, an α-amino-3-hydroxy-5-methyl-4-isoxazolepropionic acid (AMPA) receptor, a kainate receptor (KAR), and any combination thereof. Each possibility represents a separate embodiment of the invention.

In certain embodiments, the cerebral hypoxia is associated with hypoxic hypoxia, hypoxemic hypoxia, hemic hypoxia, histotoxic hypoxia, asphyctic hypoxia, hypemic hypoxia, ischemic hypoxia, ischemic stroke, hemorrhagic stroke, transient ischemic attack, acute forms thereof and/or transient forms thereof. Each possibility represents a separate embodiment of the invention.

In certain embodiments, the hypoxia is selected from the group consisting of cerebral hypoxia, diffusion hypoxia, histotoxic hypoxia, hypoxemic hypoxia, hypoxic hypoxia, intrauterine hypoxia, latent hypoxia, and any combination thereof. Each possibility represents a separate embodiment of the invention.

In certain embodiments, the neuronal damage may result in weight loss, a neurological disease or disorder, or death. In certain embodiments, the neuronal damage, if untreated, may result in weight loss, a neurological disease or disorder, or death. In certain embodiments, the neuronal damage is associated with weight loss, a neurological disease or disorder, or death. In certain embodiments, the neuronal damage may be manifested as weight loss, a neurological disease or disorder, or death. In certain embodiments, the neuronal damage, if untreated, may be manifested as weight loss, a neurological disease or disorder, or death. In certain embodiments, the neuronal damage may be manifested as weight loss, a neurological disease or disorder, or death. Each possibility represents a separate embodiment of the invention.

In certain embodiments, the subject suffers a weight loss of at least 10%, at least 20%, at least 30%, at least 40% or at least 50% compared to his weight prior to the neuronal damage. Each possibility represents a separate embodiment of the invention.

In certain embodiments, the subject suffers from a neurological disease or disorder which is known to be associated with, caused by, or already manifesting neuronal damage In certain embodiments, the subject suffers or has been diagnosed with a neurological disease or disorder selected from the group consisting of stroke, chronic cerebral ischemia, Alzheimer's disease (AD), multiple sclerosis (MS), progressive supranuclear palsy (PSP), Parkinson disease (PD), Huntington's chorea, amyotrophic lateral sclerosis, spinal trauma, brain trauma, spinal inflammation or brain inflammation. Each possibility represents a separate embodiment of the invention.

In certain embodiments, the subject is in increased risk to suffer a neuronal damage. Subjects may be associated with or diagnosed to be part of risk groups for attaining or developing neuronal damage by methods and know-how widely known in the field. In certain embodiments, the subject has experienced a condition, or is experiencing a condition, the condition selected from the group consisting of stroke, chronic cerebral ischemia, chronic cerebral hypoxia, hypoxic hypotension, cerebral hypo-perfusion and syncope. In certain embodiments, the subject has experienced a condition, or is experiencing a condition, the condition selected from the group consisting of high blood pressure, tobacco smoking, obesity, high blood cholesterol, diabetes mellitus, previous TIA, and atrial fibrillation. Each possibility represents a separate embodiment of the invention.

In certain embodiments, the subject has suffered a neuronal damage. In certain embodiments, the subject has experienced a condition selected from the group consisting of stroke, chronic cerebral ischemia, chronic cerebral hypoxia, hypoxic hypotension, cerebral hypo-perfusion and syncope. Each possibility represents a separate embodiment of the invention.

In certain embodiments, the neuronal damage is associated with at least 30% weight loss compared to his weight prior to the neuronal damage. In certain embodiments, the neuronal damage is associated with a neurological disturbance. In certain embodiments, the neuronal damage is associated with premature death. In certain embodiments, the subject has been diagnosed with stroke, chronic cerebral ischemia, Alzheimer's disease (AD), multiple sclerosis (MS), progressive supranuclear palsy (PSP), Parkinson disease (PD), Huntington's chorea, amyotrophic lateral sclerosis, spinal trauma, brain trauma, spinal inflammation or brain inflammation. In certain embodiments, the subject has been diagnosed with stroke. In certain embodiments, the subject has been diagnosed with chronic cerebral ischemia. In certain embodiments, the subject is in increased risk to suffer a neuronal damage. In certain embodiments, the subject has experienced or is experiencing a condition selected from the group consisting of stroke, chronic cerebral ischemia, chronic cerebral hypoxia, hypoxic hypotension, cerebral hypo-perfusion and syncope. In certain embodiments, the subject has experienced or is experiencing stroke. In certain embodiments, the subject has experienced or is experiencing chronic cerebral ischemia. In certain embodiments, the subject has suffered a neuronal damage. In certain embodiments, the subject has experienced a condition selected from the group consisting of stroke, chronic cerebral ischemia, chronic cerebral hypoxia, hypoxic hypotension, cerebral hypo-perfusion and syncope. Each possibility represents a separate embodiment of the invention. In certain embodiments, the subject has experienced stroke. In certain embodiments, the subject has experienced chronic cerebral ischemia.

In certain embodiments, the at least one NMDA receptor antagonist and the at least one peripheral adrenergic receptor agonist are administered in a molar ratio of at least about 50 to 1, respectively. In certain embodiments, the at least one NMDA receptor antagonist and the at least one peripheral adrenergic receptor agonist are administered in a molar ratio of at least about 100 to 1, respectively. In certain embodiments, the at least one NMDA receptor antagonist and the at least one peripheral adrenergic receptor agonist are administered in a molar ratio of at least about 125 to 1, respectively. In certain embodiments, the at least one NMDA receptor antagonist and the at least one peripheral adrenergic receptor agonist are administered in a molar ratio of at least about 250 to 1, respectively. In certain embodiments, the at least one NMDA receptor antagonist and the at least one peripheral adrenergic receptor agonist are administered in a molar ratio of at least about 500 to 1, respectively.

In certain embodiments, the at least one NMDA receptor antagonist is selected from the group consisting of an uncompetitive channel blocker, a competitive antagonist, a non-competitive antagonist, and a glycine antagonist. Each possibility represents a separate embodiment of the invention. In certain embodiments, the at least one peripheral adrenergic receptor agonist is a non-selective agonist of a plurality of adrenergic receptors. In certain embodiments, the at least one peripheral adrenergic receptor agonist is a selective agonist of a specific adrenergic receptor. In certain embodiments, the at least one peripheral adrenergic receptor agonist is a selective agonist of α1 adrenergic receptor.

In certain embodiments, the at least one peripheral adrenergic receptor agonist is selected from the group consisting of epinephrine, phenylephrine, midodrine and pseudoephedrine. Each possibility represents a separate embodiment of the invention.

In certain embodiments, the at least one peripheral adrenergic receptor agonist is epinephrine. In certain embodiments, the at least one NMDA receptor antagonist and the at least one peripheral adrenergic receptor agonist are administered in a molar ratio of about 50:1 to about 1000:1. In certain embodiments, the at least one NMDA receptor antagonist and the at least one peripheral adrenergic receptor agonist are administered in a molar ratio of about 125:1 to about 500:1. In certain embodiments, the at least one NMDA receptor antagonist and the at least one peripheral adrenergic receptor agonist are administered in a molar ratio of about 250:1 to about 500:1. In certain embodiments, the at least one NMDA receptor antagonist and the at least one peripheral adrenergic receptor agonist are administered in a molar ratio of about 125:1, about 250:1, or about 500:1. Each possibility represents a separate embodiment of the invention.

In certain embodiments, the at least one NMDA receptor antagonist and the at least one peripheral adrenergic receptor agonist are administered in a molar ratio of about 10:1 to about 200:1. In certain embodiments, the at least one NMDA receptor antagonist and the at least one peripheral adrenergic receptor agonist are administered in a molar ratio of about 25:1 to about 100:1. In certain embodiments, the at least one NMDA receptor antagonist and the at least one peripheral adrenergic receptor agonist are administered in a molar ratio of about 50:1 to about 100:1. In certain embodiments, the at least one NMDA receptor antagonist and the at least one peripheral adrenergic receptor agonist are administered in a molar ratio of about 25:1, about 50:1, or about 100:1. Each possibility represents a separate embodiment of the invention.

In certain embodiments, the at least one NMDA receptor antagonist and the at least one peripheral adrenergic receptor agonist are administered in a molar ratio of about 10:1 to about 1000:1, about 25:1 to about 500:1, or about 50:1 to about 250:1. Each possibility represents a separate embodiment of the invention.

In certain embodiments, the at least one NMDA receptor antagonist and the at least one peripheral adrenergic receptor agonist are administered in a molar ratio of about 25:1 to about 95:1, about 30:1 to about 50:1, about 45:1 to about 95:1, about 90:1 to about 180:1, about 125:1 to about 180:1, about 125:1 to about 500:1, about 130:1 to about 180:1, or about 250:1 to about 500:1. Each possibility represents a separate embodiment of the invention.

In certain embodiments, the at least one NMDA receptor antagonist is selected from the group consisting of an uncompetitive channel blocker, a competitive antagonist, a non-competitive antagonist, and a glycine antagonist. In certain embodiments, the at least one NMDA receptor antagonist is an uncompetitive channel blocker. In certain embodiments, the uncompetitive channel blocker is memantine.

In certain embodiments, the at least one peripheral adrenergic receptor agonist is selected from the group consisting of a non-selective agonist of a plurality of adrenergic receptors and a selective agonist of α1 adrenergic receptor.

In certain embodiments, the at least one peripheral adrenergic receptor agonist is a non-selective agonist of a plurality of adrenergic receptors. In certain embodiments, the at least one peripheral adrenergic receptor agonist is epinephrine.

In certain embodiments, the at least one NMDA receptor antagonist and the at least one peripheral adrenergic receptor agonist are administered in a molar ratio of about 90:1 to about 500:1. In certain embodiments, the at least one NMDA receptor antagonist and the at least one peripheral adrenergic receptor agonist are administered in a molar ratio of about 90:1 to about 180:1, about 125:1 to about 180:1, about 125:1 to about 500:1, about 130:1 to about 180:1, or about 250:1 to about 500:1. In certain embodiments, the at least one NMDA receptor antagonist and the at least one peripheral adrenergic receptor agonist are administered in a molar ratio of about 125:1 to about 180:1, about 130:1 to about 180:1, about 125:1 to about 500:1, or about 250:1 to about 500:1. In certain embodiments, the at least one NMDA receptor antagonist is memantine. Each possibility represents a separate embodiment of the invention.

In certain embodiments, the at least one peripheral adrenergic receptor agonist is a selective agonist of α1 adrenergic receptor. In certain embodiments, the at least one peripheral adrenergic receptor agonist is phenylephrine.

In certain embodiments, the at least one NMDA receptor antagonist and the at least one peripheral adrenergic receptor agonist are administered in a molar ratio of about 25:1 to about 95:1. In certain embodiments, the at least one NMDA receptor antagonist and the at least one peripheral adrenergic receptor agonist are administered in a molar ratio of about 25:1 to about 95:1, about 30:1 to about 50:1, or about 45:1 to about 95:1. In certain embodiments, the at least one NMDA receptor antagonist and the at least one peripheral adrenergic receptor agonist are administered in a molar ratio of about about 30:1 to about 50:1 or about 45:1 to about 95. In certain embodiments, the at least one NMDA receptor antagonist is memantine. Each possibility represents a separate embodiment of the invention.

In certain embodiments, the systemic administration is selected from the group consisting of oral, intraperitoneal, intramuscular, intranasal, subcutaneous, intravenous, transdermal and sublingual administration.

In certain embodiments, the systemic administration is selected from the group consisting of oral, intraperitoneal and intramuscular administration. Each possibility represents a separate embodiment of the invention.

In certain embodiments, the at least one NMDA receptor antagonist and the at least one peripheral adrenergic receptor agonist are comprised in the same pharmaceutical composition. In certain embodiments, the at least one NMDA receptor antagonist and the at least one peripheral adrenergic receptor agonist are comprised in different pharmaceutical compositions. In certain embodiments, the at least one NMDA receptor antagonist and the at least one peripheral adrenergic receptor agonist are administered at separate times. In certain embodiments, the at least one NMDA receptor antagonist and the at least one peripheral adrenergic receptor agonist are administered concomitantly.

The present invention further provides, in another aspect, a method for treating at least one symptom of transient ischemic attack or ischemic stroke in a subject in need thereof, comprising administering to the subject a pharmaceutical composition by systemic administration, the pharmaceutical composition comprising (i) at least one N-methyl-D-aspartate (NMDA) receptor antagonist, and (ii) at least one peripheral adrenergic receptor agonist, wherein the at least one NMDA receptor antagonist and the at least one peripheral adrenergic receptor agonist are administered in a molar ratio of at least about 25 to 1, respectively.

The present invention further provides, in another aspect, a method for treating at least one symptom of transient ischemic attack or ischemic stroke in a subject in need thereof, comprising administering to the subject a pharmaceutical composition by systemic administration, the pharmaceutical composition comprising: (i) at least one N-methyl-D-aspartate (NMDA) receptor antagonist, and (ii) at least one peripheral adrenergic receptor agonist, wherein the at least one NMDA receptor antagonist and the at least one peripheral adrenergic receptor agonist are administered in a molar ratio of about 25:1 to about 500:1, respectively.

The present invention further provides, in another aspect, a method for treating at least one symptom of cerebral hypoxia in a subject in need thereof, comprising administering to the subject a pharmaceutical composition by systemic administration, the pharmaceutical composition comprising (i) at least one N-methyl-D-aspartate (NMDA) receptor antagonist, and (ii) at least one peripheral adrenergic receptor agonist, wherein the at least one NMDA receptor antagonist and the at least one peripheral adrenergic receptor agonist are administered in a molar ratio of at least about 25 to 1, respectively.

The present invention further provides, in another aspect, a method for treating at least one symptom of cerebral hypoxia in a subject in need thereof, comprising administering to the subject a pharmaceutical composition by systemic administration, the pharmaceutical composition comprising: (i) at least one N-methyl-D-aspartate (NMDA) receptor antagonist, and (ii) at least one peripheral adrenergic receptor agonist, wherein the at least one NMDA receptor antagonist and the at least one peripheral adrenergic receptor agonist are administered in a molar ratio of about 25:1 to about 500:1, respectively.

In certain embodiments of the methods described above, the at least one symptom is neuronal damage.

The present invention further provides, in another aspect, a pharmaceutical composition comprising (i) at least one NMDA receptor antagonist, and (ii) at least one peripheral adrenergic receptor agonist, wherein the at least one NMDA receptor antagonist and the at least one peripheral adrenergic receptor agonist are administered in a molar ratio of at least about 25:1.

The present invention further provides, in another aspect, a pharmaceutical composition comprising: (i) at least one NMDA receptor antagonist, and (ii) at least one peripheral adrenergic receptor agonist, wherein the at least one NMDA receptor antagonist and the at least one peripheral adrenergic receptor agonist are in a molar ratio of about 25:1 to about 500:1, respectively.

Pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is considered a carrier when the pharmaceutical composition is administered as a liquid. Saline solutions and aqueous dextrose and glycerol solutions can also be employed as liquid carriers, particularly for injectable solutions.

The pharmaceutical compositions of the invention can further comprise an excipient. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfate; chelating agents such as ethylenediaminetetraacetic acid; and agents for the adjustment of tonicity such as sodium chloride or dextrose are also envisioned.

The pharmaceutical compositions of the present invention can be manufactured by processes well known in the art, e.g., by means of conventional mixing, dissolving, granulating, dragee-making, levigating, emulsifying, encapsulating, entrapping, or lyophilizing processes. Pharmaceutical compositions, which contain active ingredients, may be prepared as injectable, either as liquid solutions or suspensions, however, solid forms, which can be suspended or solubilized prior to injection, can also be prepared. The compositions can also take the form of emulsions, tablets, capsules, gels, syrups, slurries, powders, creams, depots, sustained-release formulations and the like.

Methods of introduction of a pharmaceutical composition of the invention include, but are not limited to, intravenous, subcutaneous, intramuscular, intraperitoneal, oral, topical, intradermal, intranasal, epidural, ophthalmic, and rectal routes. The pharmaceutical compositions can be administered by any convenient route, for example by infusion or bolus injection, by absorption through epithelial linings (e.g., oral mucosa, rectal and intestinal mucosa, etc.), and may be administered together with other therapeutically active agents. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

Pharmaceutical compositions for use in accordance with the present invention may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, for example in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents. Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, traditional binders and carriers such as triglycerides, microcrystalline cellulose, gum tragacanth or gelatin.

For internal applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; or a glidant such as colloidal silicon dioxide. When the dosage unit form is a capsule, it can contain, in addition to materials of the above type, a liquid carrier such as fatty oil. In addition, dosage unit forms can contain other materials which modify the physical form of the dosage unit, for example, coatings of sugar, shellac, or other enteric agents.

An active agent of the invention can be delivered in a controlled release system. For example, the active agent can be administered in combination with a biodegradable, biocompatible polymeric implant, which releases the active agent over a controlled period of time at a selected site. Examples of preferred polymeric materials include polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla.).

Pharmaceutical compositions suitable for use in the context of the present invention include compositions wherein the active ingredients are contained in an amount effective to achieve the intended purpose.

In certain embodiments, the pharmaceutical composition described above is for use in a method for preventing neuronal damage, preventing the progression of neuronal damage or treating neuronal damage in a subject in need thereof comprising administering the pharmaceutical composition to the subject by systemic administration.

In certain embodiments, the pharmaceutical composition described above is for use in a method for treating at least one symptom of transient ischemic attack or ischemic stroke in a subject in need thereof comprising administering the pharmaceutical composition to the subject by systemic administration.

In certain embodiments, the pharmaceutical composition described above is for use in a method for treating at least one symptom of cerebral hypoxia in a subject in need thereof comprising administering the pharmaceutical composition to the subject by systemic administration.

In certain embodiments, the pharmaceutical composition described above is for use in a method for preventing, ameliorating the progression or treating neuronal damage. In certain embodiments, the pharmaceutical composition described above is for use in a method for preventing, ameliorating the progression or treating at least one symptom of transient ischemic attack or ischemic stroke. In certain embodiments, the pharmaceutical composition described above is for use in a method for preventing, ameliorating the progression or treating at least one symptom of cerebral hypoxia. In certain embodiments, the use comprises administering the pharmaceutical composition by systemic administration.

The present invention further provides, in another aspect, use of a pharmaceutical composition as described above in the manufacture of a medicament.

The present invention further provides, in another aspect, a use of a pharmaceutical composition as described above in the manufacture of a medicament for (i) preventing, ameliorating the progression or treating neuronal damage, (ii) treating at least one symptom of transient ischemic attack or ischemic stroke, or (iii) treating at least one symptom of cerebral hypoxia.

The present invention further provides, in another aspect, a use of a pharmaceutical composition as described above in the manufacture of a medicament for treating at least one symptom of Alzheimer's disease (AD), multiple sclerosis (MS), progressive supranuclear palsy (PSP), Parkinson disease (PD), Huntington's chorea, amyotrophic lateral sclerosis, spinal trauma, brain trauma, spinal inflammation or brain inflammation.

The present invention further provides, in another aspect, a kit comprising: (i) at least one NMDA receptor antagonist, and (ii) at least one peripheral adrenergic receptor agonist, wherein the at least one NMDA receptor antagonist and the at least one peripheral adrenergic receptor agonist are in a molar ratio of about 25:1 to about 500:1.

The present invention provides uses of the active agents of the invention for the treatment, prophylaxis and/or inhibition of neuronal damage in a subject in need thereof, for the treatment of transient ischemic attack or ischemic stroke, and/or for the treatment of brain injury.

The present invention can be used for the treatment of ischemic conditions, for example cerebral ischemia (thromboembolic or transient ischemic attack or ischemic stroke, hemorrhage or brain injury as a result of trauma) which involve various forms of brain damage and may lead to acute or delayed damage to the brain neurons, and to neurodegeneration—for example after head trauma.

The present invention can be applicable to the treatment of relatively long-term neurodegeneration of non-ischemic origin (e.g., epilepsy, Alzheimer's disease, Huntington's disease, Downs syndrome, Multiple Sclerosis and Parkinson's disease) and neurological damage resulting from chronic infection, for example HIV producing the syndrome of AIDS.

Other conditions which can cause neuronal damage are well-known to an ordinarily skilled neurologist or similar physician and include: primary neurodegenerative disease; spinal cord lesions; hypoxic processes such as perinatal hypoxia or ischemic processes such as subsequent to cardiac arrest; neuro-trauma such as subsequent to cardiac bypass surgery or grafting; metabolically induced neurological damage; cerebral seizures; secondary neurodegenerative diseases (metabolic or toxic); memory disorders; vascular dementia, multi-infarct dementia, Lewy body dementia, or neurodegenerative dementia.

The time of treatment can be important. Administration can be before or after neuronal damage has occurred or is suspected or is anticipated. Administration before neuronal damage has occurred can be of value for prophylactic treatment, for example when the subject is considered to be at risk of an ischemic condition. Such conditions could be, for example in cardiac bypass surgery, in which a significant proportion of patients can suffer minor cerebral damage, or in childbirth, in which the fetus may be liable to problems in the fetal circulation potentially leading to anoxia and cerebral palsy and the like. Another common time of administration is after neuronal damage has occurred or is suspected, for example in the conditions of treating a stroke or a head injury, and in such cases it is desirable to make the administration as soon as possible after the event to get best results e.g. within an hour or less, though administration within several hours can still be beneficial.

According to some embodiments, the subject is a mammal. According to a certain embodiment, the mammal is a human.

Determination of a therapeutically effective amount is well within the capability of those skilled in the art, especially in light of the detailed disclosure provided herein.

The precise amount of the active agent administered to a particular subject, preferably a mammal, more preferably a human being, in the method of treatment of the present invention will depend on a number of factors, for example the specific active agent administered; its mode of administration and/or the use for which it is intended; the particular clinical condition being treated and/or its severity; and/or the age, body mass and/or past clinical history of the patient to be treated, and always lies within the sound discretion of the person administering and/or supervising the treatment, for example a medical practitioner such as nurse and/or physician. The pharmaceutical composition can contain from about 0.1% to about 99% by weight of the active agent and may prepared in unit dose form, a unit dose of an active agent generally being from about 0.1 mg to about 500 mg. Dosage amount and administration intervals may be adjusted individually to provide sufficient plasma or local levels of the active agent to induce a neuroprotective effect. Depending on the severity and responsiveness of the condition to be treated, dosing can be of a single or a plurality of administrations, with course of treatment lasting from several days to several weeks, or until cure is effected or diminution of the disease state is achieved.

FDA guidelines (Guidance for Industry, “Estimating the Maximum Safe Starting Dose in Initial Clinical Trials for Therapeutics in Adult Healthy Volunteers”, July 2005, page 7) are routinely used to convert animal doses in mg/kg to human equivalent doses in mg/kg (HED) based on body surface area, assuming a human adult weighing 60 kg and a child weighing 20 kg. In certain embodiments, dosages refer to a human adult subject weighing 60 kg. In certain embodiments, dosages correspond to the dosages of a human adult subject weighing 60 kg. The phrase “correspond to the dosages of a human adult subject weighing 60 kg” as used herein refer to a dosage which is calculated, or derived, from a dosage calculated for a 60 kg human adult subject. Such calculations/derivations are known in the art (see FDA guidelines above) and are routinely practiced by persons with average skill in the art. In certain embodiments, “dosages” refer to the dosage of a single administration. In certain embodiments, “dosages” refer to the dosage of a single administration per day. In certain embodiments, a dosage in rat in mg/kg is multiplied by a factor of about 9.7 to reach a dosage for humans in mg/kg. For example, a combination of 5 mg/kg memantine and 0.1 mg/kg phenylephrine in rats is equivalent to a combination of 48.5 mg/kg memantine and 0.97 mg/kg phenylephrine in humans Unless otherwise indicated, all dosages referred to above in mg/kg units refer to dosages in rat or mice. All of these dosages are multiplied by 9.7 to reach dosages in humans. In certain embodiments, a human patient is prophylactically or therapeutically treated to prevent neuronal damage, prevent the progression of neuronal damage or to treat neuronal damage by a combination of at least 20 mg/kg NMDA receptor antagonist and at least 0.1 mg/kg peripheral adrenergic receptor agonist.

### Definitions

As used herein to relate to ratios, e.g. molar ratios, the phrase “at least about 25 to 1” is equivalent to the phrases “at least about 25:1” and “≥22.5:1”, and relates to a ratio in which the first active agent (NMDA receptor antagonist) is at least about 25 fold more than the second active agent (peripheral adrenergic receptor agonist). The term “about” relates to a deviation of 10% from a value. For example, the phrase “about 25” means 22.5 to 27.5.

As used herein, the phrase “preventing, ameliorating the progression or treating” generally refers to any one or more of the following: delaying the onset of symptoms, reducing the severity of symptoms, reducing the severity of neuronal damage, reducing the number of symptoms, reducing the incidence of neuronal damage-related symptoms, ameliorating symptoms, reducing secondary symptoms, reducing secondary neuronal damage, prolonging patient survival, preventing relapse of neuronal damage, expediting remission, inducing remission, augmenting remission, speeding recovery, or increasing efficacy of or decreasing resistance to alternative therapeutics. As used herein, the term “symptom” generally refers to a manifestation of neuronal damage as described hereinabove. In one embodiment, “preventing or treating” refers to both therapeutic treatment and prophylactic or preventive measures, wherein the object is to prevent or lessen the neuronal damage as described hereinabove.

It will be appreciated that the term “treatment” as used herein includes both treatment and/or prophylactic use of the pharmaceutical compositions comprising the active agents of the invention. In the present invention prophylactic use of the pharmaceutical compositions comprises administering to a subject in need thereof the pharmaceutical composition to prevent the onset of neurological damage and/or to prevent the progression of neurological damage.

The active agents of the present invention have neuroprotective activity. The term “neuroprotective activity” refers to prevention of onset of neuronal damage or arresting or inhibition of progression of neuronal damage in a subject. The treatment of the present invention can be applied to a variety of acute and chronic conditions.

As used herein, the term “neuronal damage” generally refers to any injury leading to any functional neurological disability as a consequence of neuronal cell death or failure. The term “neuronal damage” includes, but is not limited to, brain infarct, brain edema, neurodegeneration, and hemorrhage.

As used herein, the term “pharmaceutical composition” refers to a preparation of one or more of the active agents described herein with other chemical components such as pharmaceutically acceptable carriers and/or excipients. The purpose of a pharmaceutical composition is to facilitate administration of an active agent to an organism. The term “pharmaceutically acceptable” means approved by a regulatory agency of the Federal or a state government or listed in the U.S. Pharmacopeia or other generally recognized pharmacopeia for use in animals, and more particularly in humans. The pharmaceutical compositions of the present invention can be formulated as pharmaceutically acceptable salts of the active agents of the present invention. The term “pharmaceutically acceptable salts” refers to salts prepared from pharmaceutically acceptable non-toxic bases or acids including inorganic or organic bases and inorganic or organic acids.

As used herein, the term “carrier” refers to a diluent or vehicle that does not cause significant irritation to an organism and does not abrogate the biological activity and properties of the administered compound. An adjuvant is included under these phrases.

As used herein, the term “excipient” refers to an inert substance added to a pharmaceutical composition to further facilitate administration of an active ingredient.

As used herein, the term “therapeutically effective amount” means an amount of an active agent effective to prevent, alleviate, or ameliorate symptoms of a condition or disease associated with neuronal damage in the subject being treated.

As used herein, the term “systemic administration” refers to a route of administration that is, e.g., enteral or parenteral, and results in the systemic distribution of an active agent leading to systemic absorption or accumulation of active agents in the blood stream followed by distribution throughout the entire body. Suitable forms, in part, depend upon the use or the route of entry, for example oral, transdermal, or by injection. Administration routes which lead to systemic absorption include, without limitations: intravenous, subcutaneous, intraperitoneal, inhalation, oral, intrapulmonary and intramuscular.

As used herein, the term “NMDA receptor antagonist” generally refers to a class of active agents that antagonize, or inhibit the action of, an N-Methyl-D-aspartate (NMDA) receptor. As used herein, the term “peripheral adrenergic receptor agonist” generally refers to a class of active agents that stimulate, or promote the action of, an adrenergic receptor.

As used herein, the phrase “is associated with” generally refers to a link between at least two variables, without being restricted to any theory or mechanism. In certain embodiments, the phrase “is associated with” means “is causing or is being caused by”. In certain embodiments, the phrase “is associated with” means “in statistically-significant linkage”.

As used herein, the term “hypoxia” generally refers to a pathological condition in which the body or a subset of cells of the body is deprived of an adequate oxygen supply. As used herein, the phrase “over-stimulation of a glutamate receptor” generally refers to a condition in which a glutamate receptor is active in a time, level and/or rate in which it causes a pathological condition.

As used herein, the term “premature death” generally refers to a death associated with the neuronal damage.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

The following examples are presented in order to more fully illustrate some embodiments of the invention. They should, in no way be construed, however, as limiting the broad scope of the invention.

### EXAMPLES

### Example 1

Anti-hypoxic effects of NMDA receptor antagonists, peripheral adrenergic receptor agonists, and combinations thereof.

Rats were intra-muscularly (I.M.) administered with 80 mg/kg sodium nitrite to achieve hemic hypoxia (Serdiuk et al., Eksp. Klin. Farmakol., 2000, Vol. 63(6), pages 3-8). Mice were caged in a 100 cm³ sealed glass chamber to achieve asphyctic hypoxia, which simulates cerebral ischemia (Serdiuk et al., Eksp. Klin. Farmakol., 2000, Vol. 63(6), pages 3-8; Khalili et al., Eur. Rev. Med. Pharmacol. Sci., 2015, Vol. 19(17), pages 3282-3285).

Animals were then treated with deionized water (control), memantine, epinephrine or combinations of memantine and epinephrine, which were I.M. or I.P administered, as indicated. Table 1 summarizes the anti-hypoxic effect of such treatments.

TABLE 1

| | | Hemic hypoxia ^{a} | Asphyctic hypoxia ^{b} |
|---|---|---|---|
| | Dose, | Rat life duration | Mice life duration |
| Treatment | mg/kg | (min), I.M. admin. | (sec), I.P. admin. |
| Control | — | 56 ± 5 | 706 ± 77 |
| Memantine | 5 | 63 ± 5 | 850 ± 80 |
| | 10 | 77 ± 8 | 936 ± 93 |
| | 20 | 86 ± 9 ¹ | 1070 ± 114 ¹ |
| Epinephrine | 0.01 | 63 ± 7 | 711 ± 79 |
| | 0.02 | 69 ± 8 | 750 ± 90 |
| | 0.1 | 113 ± 16 ² | 990 ± 123 |
| | 0.2 | 122 ± 19 ² | 1250 ± 155 ¹ |
| 1-10 mg/kg | 1.0 | 63 ± 7 | 796 ± 79 |
| Memantine + | 2.5 | 82 ± 9 ¹ | 950 ± 95 |
| 0.02 mg/kg | 5 | 102 ± 14 ¹ | 1220 ± 148 ¹ |
| epinephrine | 10 | 114 ± 17 ² | 1290 ± 158 ² |

^{a} Sodium nitrite, 80 mg/kg, I.M. administration.
^{b} Asphyxia in a sealed glass chamber (100 cm³);
¹ ρ < 0.05, compared to control (t-test Student's test);
² ρ < 0.01 compared to control (t-test).

As evident from the data presented in Table 1, only the highest dose of memantine tested alone (20 mg/kg) had a significant anti-hypoxic effect, while epinephrine was significantly effective alone in doses of 0.1 mg/kg and above. Surprisingly, when combined with a non-effective dose of 0.02 mg/kg epinephrine, memantine was significantly effective in doses of 2.5 mg/kg and above, which represents an 8-fold decrease in the minimal effective memantine dose.

### Example 2

Anti-hypoxic effects of NMDA receptor antagonists, peripheral adrenergic receptor agonists, and combinations thereof (ED50).

Rats and mice were treated as in Example 1. Table 2 summarizes the anti-hypoxic effect of such treatments.

TABLE 2

| | Hemic hypoxia ^{c} | Asphyctic hypoxia ^{d} |
|---|---|---|
| | (ED50) ^{a}, mg/kg, | (ED50) ^{b}, mg/kg, |
| Treatment | I.M. admin. | I.P. admin. |
| Control | — | — |
| Memantine | 18.2 ± 2.3 | 19.9 ± 2.8 |
| Epinephrine | 0.06 ± 0.01 | 0.12 ± 0.02 |
| Memantine + 0.02 | 2.6 ± 0.4 | 3.6 ± 0.5 |
| mg/kg epinephrine | | |
| ED50 memantine/ED50 | 7 | 5.5 |
| memantine + 0.02 | | |
| mg/kg epinephrine | | |

^{a} Dose increasing the life of rats by 50%, I.M. administration;
^{b} Dose increasing the life of mice by 50%, I.P. administration;
^{c} Sodium nitrite, 80 mg/kg, I.M. administration;
^{d} Asphyxia in a sealed glass chamber (100 cm³).

As evident from the data presented in Table 2, ED50 of memantine was 18-20 mg/kg, while ED50 of epinephrine was 0.06-0.12 mg/kg. Surprisingly, when combined with a non-effective dose of 0.02 mg/kg epinephrine, the ED50 of memantine was lowered to 2.6-3.6 mg/kg, which represents a 5.5 to 7-fold decrease in the ED50 of memantine.

### Example 3

Neuroprotective effects of NMDA receptor antagonists, peripheral adrenergic receptor agonists, and combinations thereof.

Mice were I.P. administered with 250 mg/kg N-Methyl-D-aspartic acid (NMDA), a neurotoxin causing clonic-tonic seizure and death in 100% of animals by toxic stimulating of NMDA receptors of brain (Leander et al., Brain Res., 1988, Vol. 448 (1), pages 115-120). Rats were I.M. administered with 12 mg/kg Kainic acid to achieve chronic kainate degeneration of the brain by stimulation of AMPA/kainate receptors (Serdyuk et al., Bull. Exp. Biol. Med., 2014, Vol. 157(1), pages 15-17). Chronic kainate degeneration leads to chronic kainate lethality, neurologic disturbances and large weight loss (Serdyuk et al., Bull. Exp. Biol. Med., 2014, Vol. 157(1), pages 15-17).

Animals were then treated with deionized water (control), memantine, epinephrine or combinations of memantine and epinephrine, which were I.P. or I.M administered, as indicated. Table 3 summarizes the neuroprotective effect of such treatments.

TABLE 3

| | | Acute NMDA | Chronic Kainate degeneration ^{b}, I.M. admin. | | |
|---|---|---|---|---|---|
| | | lethality in | Chronic Kainate | Rats with | Rats with |
| | Dose, | mice ^{a} (%), | lethality in | heavy neurologic | large weight |
| Treatment | mg/kg | I.P. admin. | rats ^{c} (%) | disturbances ^{d} (%) | loss ^{e} (%) |
| Control | | 100 | 90 | 100 | 100 |
| Memantine | 5 | 100 | 90 | 90 | 90 |
| | 10 | 60 | 70 | 80 | 70 |
| | 20 | 16 ¹ | 40 ¹ | 50 ¹ | 40 ¹ |
| Epinephrine | 0.01 | 100 | 90 | 100 | 100 |
| | 0.02 | 100 | 80 | 90 | 80 |
| | 0.1 | 30 ¹ | 20 ¹ | 30 ¹ | 20 ² |
| | 0.2 | 10 ² | 0 ² | 10 ² | 0 ² |
| 1-10 mg/kg | 1.0 | 90 | 70 | 80 | 70 |
| Memantine + | 2.5 | 80 | 40 ¹ | 50 ¹ | 40 ¹ |
| 0.02 mg/kg | 5 | 20 ¹ | 10 ² | 20 ¹ | 10 ² |
| epinephrine | 10 | 0 ¹ | 0 ² | 0 ² | 0 ² |

^{a} NMDA 250 mg/kg, I.P. administration;
^{b} Kainic acid 12 mg/kg, I.M. administration;
^{c} 2 weeks after the administration of kainate;
^{d} Neurological disorders score from 3 to 5 points, assessed 7-14 days after injection of kainate;
^{e} Underweight more than 30% compared to baseline, assessed 7-14 days after injection of kainate;
¹ ρ < 0.05, compared to control (F-test Fisher's test);
² ρ < 0.01 compared to control (F-test).

As evident from the data presented in Table 2, only the highest dose of memantine tested alone (20 mg/kg) had a significant neuroprotective effect, while epinephrine was significantly effective alone in doses of 0.1 mg/kg and above. Surprisingly, when combined with a non-effective dose of 0.02 mg/kg epinephrine, memantine was significantly effective in doses of 2.5 mg/kg and above, which represents a 4 to 8-fold decrease in the minimal effective memantine dose.

### Example 4

Neuroprotective effects of NMDA receptor antagonists, peripheral adrenergic receptor agonists, and combinations thereof (ED50).

Rats were treated as in Example 3. Table 4 summarizes the neuroprotective effect of such treatments.

TABLE 4

| | | Chronic kainate degeneration ^{b}, I.M. admin. | | |
|---|---|---|---|---|
| | Dose reducing acute | Dose reducing | Dose reducing the | Dose reducing the |
| | NMDA mortality in | Chronic kainate | number of rats with | number of rats with |
| | mice ^{a} by 50% | lethality in | severe neurological | a large deficit of |
| | (ED 50) mg/kg, | rats ^{c} by 50% | disorders ^{d} by 50% | weight ^{e} by 50% |
| Treatment | I.P. admin. | (ED50), mg/kg | (ED50), mg/kg | (ED50), mg/kg |
| Control | — | — | — | — |
| Memantine | 12.6 ± 1.8 | 17.7 ± 2.3 | 20.0 ± 2.5 | 17.6 ± 2.4 |
| Epinephrine | 0.07 ± 0.01 | 0.05 ± 0.007 | 0.07 ± 0.01 | 0.05 ± 0.008 |
| Memantine + | 3.6 ± 0.5 | 1.8 ± 0.3 | 2.5 ± 0.4 | 1.8 ± 0.2 |
| 0.02 mg/kg | | | | |
| epinephrine | | | | |
| ED50 memantine/ | 3.5 | 9.8 | 8 | 9.7 |
| ED50 memantine + | | | | |
| 0.02 mg/kg | | | | |
| epinephrine | | | | |

^{a} NMDA 250 mg/kg, I.P. administration,
^{b} Kainic acid 12 mg/kg, IM administration;
^{c} 2 weeks after the administration of kainate;
^{d} Neurological disorders score from 3 to 5 points, assessed 7-14 days after injection of kainate;
^{e} Underweight more than 30% compared to baseline, assessed 7-14 days after injection of kainate.

As evident from the data presented in Table 4, ED50 of memantine was 12-20 mg/kg, while ED50 of epinephrine was 0.05-0.07 mg/kg. Surprisingly, when combined with a non-effective dose of 0.02 mg/kg epinephrine, the ED50 of memantine was lowered to 1.8-3.6 mg/kg, which represents a 3.5 to 10-fold decrease in the ED50 of memantine.

### Example 5

Anti-hypoxic and neuroprotective effects of NMDA receptor antagonists, peripheral adrenergic receptor agonists, and combinations thereof.

Rats were either orally administered with 80 mg/kg sodium nitrite to achieve hemic hypoxia, as explained In Example 1, or with 12 mg/kg Kainic acid to achieve chronic kainate degeneration of the brain by stimulation of AMPA/kainate receptors. Rats were then treated with deionized water (control), memantine, phenylephrine or combinations of memantine and phenylephrine, which were orally administered 40 minutes before sodium nitrite or kainic acid administration, as indicated. Table 5 summarizes the anti-hypoxic and neuroprotective effects of such treatments.

TABLE 5

| | | Hemic hypoxia ^{a} | Chronic kainate degeneration ^{b}, Oral admin. | | |
|---|---|---|---|---|---|
| | | Life of rats | Chronic kainate | Rats with heavy | Rats with |
| | Dose, | (min), Oral | lethality in rats ^{c}, | neurological | large weight |
| Treatment | mg/kg | admin. | % per group | diseases ^{d} (%) | loss ^{e} (%) |
| Control | — | 56 ± 5 | 90% | 100% | 100% |
| Memantine | 5 | 62 ± 7 | 90% | 90% | 90% |
| | 10 | 65 ± 9 | 80% | 80% | 70% |
| | 20 | 73 ± 11 | 60% ³ | 70% | 60% ³ |
| Phenylephrine | 0.02 | 60 ± 7 | 90% | 100% | 100% |
| | 0.05 | 62 ± 9 | 90% | 90% | 90% |
| | 0.1 | 67 ± 10 | 80% | 80% | 70% |
| | 0.5 | 77 ± 12 | 40% ³ | 50% ³ | 40% ³ |
| | 1.0 | 88 ± 14 ¹ | 20% ³ | 20% ³ | 10% ⁴ |
| 1-10 mg/kg | 1.0 | 63 ± 7 | 80% | 80% | 70% |
| Memantine + | 2.5 | 75 ± 9 | 60% ³ | 70% | 60% |
| 0.1 mg/kg | 5 | 94 ± 12 ¹ | 40% ³ | 50% ³ | 40% ³ |
| phenylephrine | 10 | 97 ± 15 ² | 20% ³ | 20% ³ | 10% ⁴ |

^{a} sodium nitrite, 80 mg/kg;
^{b} Kainic acid 12 mg/kg;
^{c} 2 weeks after the administration of kainate;
^{d} Neurological disorders score from 3 to 5 points, assessed 7-14 days after injection of kainate;
^{e} Underweight more than 30% compared to baseline, assessed 7-14 days after administration of kainate;
¹ ρ < 0.05 compared to control (t-test Student's test);
² ρ < 0.01 compared to control (t-test);
³ ρ < 0.05 compared to control (F-test, Fisher' test);
⁴ ρ < 0.01 compared to control (F-test).

As evident from the data presented in Table 5, only the highest dose of memantine tested (20 mg/kg) had some significant neuroprotective effects, while phenylephrine was significantly effective in doses of 0.5 mg/kg and above. Surprisingly, when combined with a non-effective dose of 0.1 mg/kg phenylephrine, memantine was significantly effective in doses of 2.5 mg/kg and above, which represents an 8-fold decrease in the effective memantine dose.

### Example 6

Anti-hypoxic and neuroprotective effects of NMDA receptor antagonists, peripheral adrenergic receptor agonists, and combinations thereof (ED50).

Rats were treated as in Example 5. Table 6 summarizes the neuroprotective effect of such treatments.

TABLE 6

| | Hemic hypoxia ^{a} | Chronic kainate degeneration^{a}, Oral admin. | | |
|---|---|---|---|---|
| | Dose increasing | Dose decreasing | Dose decreasing | Dose decreasing the |
| | the life of rats | chronic kainate | heavy neurological | number of rats with |
| | by 50% (ED50), | lethality ^{b} by 50% | diseases ^{c} by 50% | large weight loss ^{d} |
| Treatment | mg/kg, Oral admin. | (ED50), mg/kg | (ED50), mg/kg | by 50% (ED50), mg/kg |
| Memantine | 19.6 ± 1.9 | 18.7 ± 2.3 | 20.0 ± 2.5 | 18.8 ± 2.4 |
| Phenylephrine | 0.8 ± 0.1 | 0.4 ± 0.06 | 0.05 ± 0.08 | 0.04 ± 0.07 |
| Memantine + | 3.6 ± 0.6 | 3.5 ± 0.5 | 5.0 ± 0.7 | 3.5 ± 0.4 |
| 0.1 mg/kg | | | | |
| phenylephrine | | | | |
| ED50 Memantine/ | 5.4 | 5.3 | 4 | 5.4 |
| ED50 Memantine + | | | | |
| 0.1 mg/kg | | | | |
| phenylephrine | | | | |

^{a} Kainic acid 12 mg/kg I.M.;
^{b} 2 weeks after the administration of kainate;
^{c} Neurological disorders score from 3 to 5 points, assessed 7-14 days after injection of kainate;
^{d} Underweight more than 30% compared to baseline, assessed 7-14 days after administration of kainate.

As evident from the data presented in Table 6, ED50 of memantine alone was 18-20 mg/kg, while ED50 of phenylephrine alone was 0.04-0.8 mg/kg. Surprisingly, when combined with a non-effective dose of phenylephrine (0.1 mg/kg), the ED50 of memantine was lowered to 3.5-5 mg/kg, which represents a 4 to 5.4-fold decrease in the ED50 of memantine.

It is important to note that the scope of the invention is not construed as being limited by the illustrative embodiments set forth herein. Other variations are possible within the scope of the present invention as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

1-43. (canceled)

44. A method for preventing, ameliorating the progression or treating neuronal damage in a subject in need thereof, the method comprising systemically administering to the subject:
(i) at least one N-methyl-D-aspartate (NMDA) receptor antagonist, and
(ii) at least one peripheral adrenergic receptor agonist,
wherein the at least one NMDA receptor antagonist and the at least one peripheral adrenergic receptor agonist are administered in a molar ratio of about 25:1 to about 500:1, respectively.

45. The method of claim 44, wherein the neuronal damage is associated with cerebral hypoxia, cerebral ischemia, or over-stimulation of an ionotropic neuronal glutamate receptor.

46. The method of claim 45, wherein the ionotropic neuronal glutamate receptor is selected from the group consisting of an NMDA receptor, an α-amino-3-hydroxy-5-methyl-4-isoxazolepropionic acid (AMPA) receptor, a kainate receptor (KAR), and any combination thereof.

47. The method of claim 44, wherein the subject has been diagnosed with stroke, chronic cerebral ischemia, Alzheimer's disease (AD), multiple sclerosis (MS), progressive supranuclear palsy (PSP), Parkinson disease (PD), Huntington's chorea, amyotrophic lateral sclerosis, spinal trauma, brain trauma, spinal inflammation or brain inflammation.

48. The method of claim 44, wherein the subject has experienced a condition selected from the group consisting of stroke, chronic cerebral ischemia, chronic cerebral hypoxia, hypoxic hypotension, cerebral hypo-perfusion and syncope.

49. The method of claim 44, wherein the at least one NMDA receptor antagonist and the at least one peripheral adrenergic receptor agonist are administered in a molar ratio of about 25:1 to about 95:1, about 30:1 to about 50:1, about 45:1 to about 95:1, about 90:1 to about 180:1, about 125:1 to about 180:1, about 125:1 to about 500:1, about 130:1 to about 180:1, or about 250:1 to about 500:1.

50. The method of claim 44, wherein the at least one NMDA receptor antagonist is selected from the group consisting of an uncompetitive channel blocker, a competitive antagonist, a non-competitive antagonist, and a glycine antagonist.

51. The method of claim 50, wherein the uncompetitive channel blocker is memantine.

52. The method of claim 44, wherein the at least one peripheral adrenergic receptor agonist is selected from the group consisting of a non-selective agonist of a plurality of adrenergic receptors and a selective agonist of α1 adrenergic receptor.

53. The method of claim 52, wherein the at least one peripheral adrenergic receptor agonist is a non-selective agonist of a plurality of adrenergic receptors, and wherein the non-selective agonist of a plurality of adrenergic receptors is epinephrine.

54. The method of claim 52, wherein the at least one peripheral adrenergic receptor agonist is a selective agonist of α1 adrenergic receptor, and wherein the selective agonist of α1 adrenergic receptor is phenylephrine.

55. The method of claim 44, wherein the at least one NMDA receptor antagonist and the at least one peripheral adrenergic receptor agonist are administered in a molar ratio of about 25:1 to about 95:1.

56. The method of claim 44, wherein the systemic administration is selected from the group consisting of oral, intraperitoneal and intramuscular administration.

57. The method of claim 44, wherein the at least one NMDA receptor antagonist and the at least one peripheral adrenergic receptor agonist are comprised in the same pharmaceutical composition.

58. The method of claim 44, wherein the at least one NMDA receptor antagonist and the at least one peripheral adrenergic receptor agonist are comprised in different pharmaceutical compositions.

59. The method of claim 44, wherein the at least one NMDA receptor antagonist and the at least one peripheral adrenergic receptor agonist are administered at separate times or concomitantly.

60. A method for treating at least one symptom of transient ischemic attack or ischemic stroke in a subject in need thereof, comprising administering to the subject a pharmaceutical composition by systemic administration, the pharmaceutical composition comprising:
(i) at least one N-methyl-D-aspartate (NMDA) receptor antagonist, and
(ii) at least one peripheral adrenergic receptor agonist,
wherein the at least one NMDA receptor antagonist and the at least one peripheral adrenergic receptor agonist are administered in a molar ratio of about 25:1 to about 500:1, respectively.

61. The method of claim 60, wherein the at least one symptom is neuronal damage.

62. A pharmaceutical composition comprising:
(i) at least one NMDA receptor antagonist, and
(ii) at least one peripheral adrenergic receptor agonist,
wherein the at least one NMDA receptor antagonist and the at least one peripheral adrenergic receptor agonist are in a molar ratio of about 25:1 to about 500:1, respectively.
